(19) United States
(12) Patent Application Publication
(10) Pub. No.: US 2026/0335224 A1
(43) Pub. Date: Oct. 29, 2026

(54) TREATMENT WITH POWDERED INTRANASAL EPINEPHRINE

(71) Applicant: Nasus Pharma Ltd., Tel Aviv (IL)

(72) Inventors: Galia TEMTSIN-KRAYZ, Ashdod (IL); Pavel KAZHDAN, Yavne (IL)

(21) Appl. No.: 19/737,315

(22) Filed: Jul. 10, 2026

Related U.S. Application Data
(63) Continuation-in-part of application No. 17/911,523, filed on Mar. 16, 2021, now Pat. No. 12,678,402.
Application No. 17/911,523 is a continuation of application No. 17/135,528, filed on Dec. 28, 2020, now Pat. No. 11,400,045.
(60) Provisional application No. 62/989,913, filed on Mar. 16, 2020.
(60) Provisional application No. 62/989,913, filed on Mar. 16, 2020.

(51) Int. Cl. A61K 9/00; A61K 9/14; A61K 9/16; A61K 31/137

(52) U.S. Cl. CPC A61K 9/0043; A61K 9/143; A61K 9/145; A61K 9/1623; A61K 31/137

(57) ABSTRACT

Disclosed is a pharmaceutical composition in dry powder form for intranasal administration, comprising an anti-anaphylactic adrenergic receptor agonist in the form of dry powder for intranasal administration, the composition comprising solid particles of the active agent in combination with at least one functional additive, and solid particles of an inert carrier.

### CROSS-REFERENCE TO RELATED APPLICATIONS

This application is a continuation-in-part of U.S. application Ser. No. 17/911,523, filed Sep. 14, 2022, now allowed, which is a U.S. National Stage application under 35 U.S.C. § 371 of International Application PCT/IL2021/050288, filed Mar. 16, 2021, which is a continuation of U.S. application Ser. No. 17/135,528, filed Dec. 28, 2020, now U.S. Pat. No. 11,400,045, which claims the benefit of priority to U.S. provisional application 62/989,913. PCT/IL2021/050288 also claims benefit to U.S. provisional application 62/989,913. Benefit of the filing date of each of these prior applications is hereby claimed. Each of these prior applications is hereby incorporated by reference in their entirety.

### TECHNICAL FIELD

Disclosed are dry powder compositions for intranasal administration of adrenergic receptor agonists, methods for their preparation and uses thereof in medical treatment.

### PRIOR ART

Several publications referred to herein are indicated by Arab numerals in parentheses. These publications may be considered relevant as background for the presently disclosed subject matter. A full list of these publications appears at the end of the description, immediately preceding the claims.

### BACKGROUND

### Intranasal Drug Delivery

Intranasal delivery has a number of compelling advantages over other routes of administration, namely its non-invasiveness, rapid attainment of therapeutically relevant concentrations to the bloodstream, no first-pass metabolism, and ease of administration. Viable nasal delivery technologies are expected to enable the development of innovative pharmaceutical formulations and medicaments of novel as well as approved active pharmaceuticals ingredients by delivery via novel routes of administration.

The intranasal delivery of drugs utilizes devices of several types, such as nebulizers, pressurized devices, dry powder sprayers, and bi-directional nasal devices.

Anaphylaxis is a systemic and life-threatening allergic reaction characterized by anaphylactic shock associated with a critical decrease in blood pressure and deterioration in consciousness. The most frequent triggers of severe anaphylactic reactions are drugs, insect venoms, and foods [(1), (2)]. Epinephrine is currently a universally recommended as the initial drug of choice for the treatment of anaphylaxis [(3), (4), (5)], providing a unique effect on body systems potentially involved in anaphylaxis.

### Epinephrine

Epinephrine acts on both α- and β-adrenergic receptors. Through its action on α-adrenergic receptors, epinephrine decrease the vasodilation and increased vascular permeability that occurs during anaphylaxis, which can lead to loss of intravascular fluid volume and hypotension. Through its action on β-adrenergic receptors, epinephrine causes bronchial smooth muscle relaxation and helps alleviate bronchospasm, wheezing and dyspnea that may occur during anaphylaxis. Epinephrine is also known to alleviate pruritus, urticaria, and angioedema and may relieve gastrointestinal and genitourinary symptoms associated with anaphylaxis because of its relaxant effects on the smooth muscle of the stomach, intestine, uterus, and urinary bladder. Through the activation of α- and β-adrenergic receptors, epinephrine functionally antagonizes all of the important pathomechanisms of anaphylaxis by vasoconstriction, reduction of vascular permeability, bronchodilatation, edema reduction, and positive inotropy in the heart (6).

Epinephrine is currently available for use in only an injectable dosage form, in ampules or in auto-injectors (7). Each auto-injector contains a single dose of epinephrine and the recommended dose is 0.3 mg/0.3 mL or 0.5 mg/ml epinephrine injection for single-use. Repeat injections may be needed with severe persistent anaphylaxis.

Failure to administer epinephrine promptly has been identified as the most important factor contributing to death in patients with systemic anaphylaxis. Hence, it is recommended that patients with history of severe anaphylactic reactions or the caregivers have epinephrine injection readily available for first aid treatment (8). The use of epinephrine injection is however limited due to its potential drawbacks. Research has been conducted to find alternatives to epinephrine auto-injectors. The feasibility of epinephrine administration via intranasal (IN) route in humans has been demonstrated in clinical studies. In emergency situations, intranasal delivery could be considered as an alternative route of epinephrine injection due to the convenience in drug administration.

When given subcutaneously or intramuscularly, epinephrine has a rapid onset and short duration of action.

The results of a recent study (INS015-17-112) comparing two formulations of epinephrine nasal spray, aqueous (AQ) and hydro-alcoholic (HA) (both investigational products were developed by Insys) to EpiPen® in adults with seasonal allergy reported that epinephrine was rapidly absorbed following single 6 mg AQ and HA doses vs. EpiPen® 0.3 mg IM, with epinephrine plasma concentrations above 100 μg/mL within 5 minutes and median Tₘₐₓ within 5-16 minutes for AQ, 3-10 minutes for HA, and 5 minutes for EpiPen®. Allergen challenge mainly impacted epinephrine absorption, with Cₘₐₓ increased 1.72-fold for AQ and 1.43-fold for HA, with minor change in AUC for AQ or HA; EpiPen® exposure was unaffected (9).

Similarly, another study in healthy volunteers showed that intranasal epinephrine at 5 mg in saline formulation had significant systemic absorption which was comparable to IM epinephrine 0.3 mg with the average area under the curve (AUC) values at 0-120 minutes of 18.3 and 19.4 ng*min/mL, respectively (9).

The pharmacokinetic (PK) characteristics of epinephrine from the literature are presented in Table 1 (9):

TABLE 1
Pharmacokinetic Characteristics of Epinephrine
via Intramuscular and Intranasal routes

| | | Cₘₐₓ | C_{baseline} | |
|---|---|---|---|---|
| | | (pg/mL) | (pg/mL) | AUC₀₋₁₂₀ |
| Epinephrine | Tₘₐₓ | Mean | Mean | min |
| formulation | (min) | (±SD) | (±SD) | (ng · min/mL) |
| Epinephrine 0.3 mg IM | 67 ± 43 | 309 ± 88 | 35 ± 23 | 18.3 ± 9.3 |
| Epinephrine 5 mg IN | 70 ± 17 | 386 ± 152 | 8 ± 6 | 19.4 ± 12.1 |

Dry powders are used in intranasal drug delivery due to the many advantages of using this dosage form including the improved stability, administration of larger doses and lack of microbial growth (no need for preservatives). The administration of intranasal powders may improve patient compliance, especially where the smell and taste of the delivered solution composition comprising excipients is unpleasant. Compared to drug solutions, the administration of powders can result in a prolonged contact with the nasal mucosa. Powder form is suitable for delivery of both small molecules and biologicals, especially peptides, hormones and antibodies.

WO2019/038756 describes a pharmaceutical composition in a form of dry powder for intranasal administration, the composition comprising solid particles of at least one active agent and solid particles of a carrier/disaggregation agent/deagglomerating agent/diluent, the pharmaceutical composition being substantially free of excipients other than the solid diluent, the pharmaceutical composition having at least 90% of the particles of the active agent with a mean particle size of 10-30 microns and less than 10% of the particles of the active agent with a mean particle size equal to or below 5 microns, and having the particles of said disaggregation agent/diluent with a mean particle size of 50-200 microns.

### SUMMARY OF INVENTION

Disclosed herein is a pharmaceutical composition comprising as active agent an anti-anaphylactic adrenergic receptor agonist in the form of dry powder for intranasal administration, said composition comprising a first type of solid particles comprising at least one active agent in combination with at least one functional additive, and a second type of solid particles comprising a pharmaceutically acceptable carrier/diluent/disaggregating/deagglomerating agent, wherein at least about 90% of said first type particles are of a mean particle size of about 10-30 microns and less than about 10% of said first type particles are of a mean particle size equal to or below about 10 microns and said second type particles are of a mean particle size greater than that of the first type particles. The second type particles are of a mean particle size of about 50-200 microns.

In all aspects and embodiments of the present disclosure, the pharmaceutical composition of can be substantially free of excipients other than said at least one functional additive comprised in said first type particles and said carrier comprised in said second type particles.

In embodiments of the disclosed pharmaceutical composition the active agent can be any one of epinephrine, norepinephrine, dopamine or antihistamine or pharmaceutically acceptable salts or derivatives thereof, but is not limited thereto. In some specific embodiments, the active agent is epinephrine or a pharmaceutically acceptable salt thereof, which can be any one of pharmaceutically acceptable bitartrate, hydrochloride or borate salt thereof, as well as hydrates and anhydrates thereof.

The functional additive comprised in said first type particles can be any one of a buffering agent, glidant or lubricant. The buffering agent comprised in said first type particles can be sodium di-hydrogen phosphate, but is not limited thereto.

In embodiments of the presently disclosed pharmaceutical composition the ratio between the at least one pharmaceutically active agent and the at least one functional additive in the first type particles is predetermined, according to chemical and other properties of the specific constituents.

In embodiments of the presently disclosed pharmaceutical composition the carrier/diluent/disaggregating/deagglomerating agent can be any one of lactose monohydrate, lactose, a lactose functional analogue, or any mixture of at least two thereof. Alternatively, the carrier/diluent/disaggregating/deagglomerating agent can be any one of dextrose, sorbitol, mannitol, maltitol and xylitol, a cellulose or cellulose derivative, or starch or starch derivative.

In embodiments of the presently disclosed pharmaceutical composition the weight ratio between said first type particles and said second type particle can be between 1:9 to 9:1, for example 1:9, 2:8, 3:7, 4:6, 5:5, 6:6, 7:3, 8:2 or 9:1, and any mid-ratios therebetween.

In a specific embodiment, the present disclosure provides an epinephrine pharmaceutical composition in the form of dry powder for intranasal administration, comprising as active agent epinephrine or a pharmaceutically acceptable salt thereof, said composition comprising a first type of solid particles comprising epinephrine or a pharmaceutically acceptable salt thereof in combination with a physiologically acceptable buffering agent, and a second type of solid particles comprising lactose monohydrate as carrier, wherein at least about 90% of said first type particles are of a mean particle size of about 10-30 microns and less than about 10% of said first type particles are of a mean particle size equal to or below about 10 microns and said second type particles are of a mean particle size greater than that of the first type particles, providing a metered therapeutically effective nominal dose of said epinephrine or pharmaceutically acceptable salt thereof. In this epinephrine pharmaceutical composition the molar ratio between the epinephrine bitartrate to sodium dihydrogen phosphate can be 0.9:1. The therapeutically effective amount of epinephrine in this epinephrine pharmaceutical composition is essentially equivalent to about 0.3 mg or 0.5 mg epinephrine administered i.m. (intra-muscularly, also referred to as IM).

Further disclosed herein is a disposable dose unit form for intranasal administration to a subject of a pharmaceutical composition according to any one of claims 1 to 12, wherein said dose unit is loaded with a predetermined single dose of the composition and provides the subject with a metered dose the pharmaceutically active adrenergic receptor agonist. The disposable dose unit form can be loaded with a predetermined single dose of the composition and provides the subject with a metered dose epinephrine.

In some embodiments, the dose unit is loaded with a predetermined single dose of the composition and provides the subject with a metered dose epinephrine being equivalent to about 0.3 mg or 0.5 mg epinephrine administered intramuscularly.
a. In a further aspect disclosed herein is a kit for intranasal administration of epinephrine comprising at least one dose unit for intranasal administration comprising a pharmaceutical composition as disclosed herein and instructions for use.

Still further, provided herein is a method of treating anaphylactic shock in a patient in need thereof, said method comprising administering to said patient a therapeutically effective amount of a composition as disclosed herein or at least one dose unit as disclosed herein.

In a specific embodiment, the present disclosure provides a method of treating anaphylactic shock in a patient in need thereof, said method comprising administering to said patient a therapeutically effective amount of an epinephrine composition as defined herein or at least one epinephrine dose unit as disclosed herein.

In accordance with some aspects, the present disclosure provides a dry powder pharmaceutical composition for intranasal administration comprising epinephrine or a pharmaceutically acceptable salt thereof and a pharmaceutically acceptable carrier, wherein the pharmaceutical composition is characterized in that the composition, when emitted from an intranasal powder delivery device, has an emitted particle size distribution with an emitted Dv90 of less than about 400 microns.

In accordance with some other aspects, the present disclosure provides a dry powder pharmaceutical composition for intranasal administration comprising epinephrine or a pharmaceutically acceptable salt thereof and a pharmaceutically acceptable carrier, wherein the pharmaceutical composition is characterized in that the composition, when emitted from an intranasal powder delivery device, has an emitted particle size distribution that is essentially the same as a bulk particle size distribution.

In accordance with some further aspects, the present disclosure provides a dry powder pharmaceutical composition for intranasal administration comprising epinephrine or a pharmaceutically acceptable salt thereof and a pharmaceutically acceptable carrier, wherein the pharmaceutical composition is characterized in that the composition, when emitted from an intranasal powder delivery device, has an emitted particle size distribution with an emitted Dv50 between about 25 microns and about 200 microns and an emitted Dv10 between about 5 microns and about 25 microns.

In accordance with yet some other aspects, the present disclosure provides a dry powder pharmaceutical composition for intranasal administration comprising epinephrine or a pharmaceutically acceptable salt thereof and a pharmaceutically acceptable carrier, wherein the pharmaceutical composition is characterized in that the composition is substantially free of agglomerated particles and retains at least 80% of initial drug assay after storage for 3 months at 40 °C with 75% relative humidity.

In accordance with yet some further aspects, the present disclosure provides a method of treating anaphylaxis or anaphylactic shock in a subject in need thereof, comprising intranasally administering to the subject a therapeutically effective amount of the dry powder pharmaceutical composition as described herein.

In accordance with some other aspects, the present disclosure provides a method of intranasally delivering epinephrine to a subject in need thereof, comprising actuating an intranasal powder delivery device comprising the dry powder pharmaceutical composition as described herein.

In some embodiments, the dry powder pharmaceutical compositions applicable by the methods described herein comprises epinephrine or a pharmaceutically acceptable salt thereof and a pharmaceutically acceptable carrier, wherein the pharmaceutical composition is characterized in that the composition, when emitted from an intranasal powder delivery device, has an emitted particle size distribution with an emitted Dv90 of less than about 400 microns.

In some embodiments, the dry powder pharmaceutical compositions applicable by the methods described herein comprises epinephrine or a pharmaceutically acceptable salt thereof and a pharmaceutically acceptable carrier, wherein the pharmaceutical composition is characterized in that the composition, when emitted from an intranasal powder delivery device, has an emitted particle size distribution that is essentially the same as a bulk particle size distribution.

In some embodiments, the dry powder pharmaceutical compositions applicable by the methods described herein comprises epinephrine or a pharmaceutically acceptable salt thereof and a pharmaceutically acceptable carrier, wherein the pharmaceutical composition is characterized in that the composition, when emitted from an intranasal powder delivery device, has an emitted particle size distribution with an emitted Dv50 between about 25 microns and about 200 microns and an emitted Dv10 between about 5 microns and about 25 microns.

In some embodiments, the dry powder pharmaceutical compositions applicable by the methods described herein comprises epinephrine or a pharmaceutically acceptable salt thereof and a pharmaceutically acceptable carrier, wherein the pharmaceutical composition is characterized in that the composition is substantially free of agglomerated particles and retains at least 80% of initial drug assay after storage for 3 months at 40 °C with 75% relative humidity.

### BRIEF DESCRIPTION OF THE DRAWINGS

In order to better understand the subject matter that is disclosed herein and to exemplify how it can be carried out in practice, embodiments will now be described, by way of non-limiting example only, with reference to the accompanying drawings, in which:

FIG. 1 exemplary Unit Dose Powder Device and its components for nasal administration

FIG. 2 schematic representation of the modified spray-dryer apparatus used in the present examples, as described in Example 1

FIG. 3 SEM images of lactose monohydrate (large irregular particles) and epinephrine bitartrate/sodium di-hydrogen phosphate particles (small spherical particles) of the dry powder intranasal formulation obtained in Example 2.

FIG. 4 shows the particle size distribution of the dry powder intranasal formulation obtained in Example 2.

FIG. 5 shows the particle size distribution of particles emitted from a device.

### DESCRIPTION OF EMBODIMENTS

Disclosed herein are novel formulations the form of dry powder, for intranasal administration of pharmaceutically active agent/s. Generally, a formulation according to the present disclosure comprises two types of solid particles, a first type of essentially spherical particles comprising the pharmaceutically active agent in combination with a functional additive, and a second type of irregularly shaped particles comprising an essentially inert carrier/diluent/disaggregating/deagglomerating agent.

In a first aspect, the present disclosure relates to a pharmaceutical composition in the form of dry powder for intranasal administration, comprising a first type of solid particles comprising at least one pharmaceutically active agent in combination at least one functional additive, and a second type of solid particles comprising a pharmaceutically acceptable carrier/diluent/disaggregating/deagglomerating agent, in which at least about 90% of said first type particles are of a mean particle size of about 10-30 microns and less than about 10% of said first type particles are of a mean particle size equal to or below about 10 microns and said second type particles are of a mean particle size greater than that of the first type particles, such as a mean particle size of about 50-200 microns.

Active agents for intranasal administration in dry powder form are usually produced by milling techniques. As a result, their particle size distribution is broad and the particles are usually non-spherical and non-uniform. The presence of active agent particles of less than 5 microns (μm) should however be avoided. Such very small particles may reach the lung mucosa by nasal spraying or by inhaling, which completely unacceptable for intranasal administration from the safety point of view. Therefore, the size distribution of the presently disclosed compositions, wherein the major part (about 90%) of the particles comprising the pharmaceutically active agent have a mean size of 10-30 microns, and only less than 10% of the particles are of a mean diameter of less than 5 microns their use in nasal spraying renders them beneficial for the intranasal administration.

In all embodiments of all aspects of the present disclosure, the pharmaceutically active agent can be an adrenergic receptor agonist, for example, but not limited to any one of epinephrine, norepinephrine, dopamine or antihistamine or pharmaceutically acceptable salts or derivatives thereof. A specific pharmaceutically active agent is, but not limited to, epinephrine or a pharmaceutically acceptable salt thereof, such as, but not limited to any one of bitartrate, hydrochloride or borate salts of epinephrine.

In all embodiments of all aspects of the present disclosure, the said functional additive can be any one of a buffering agent, glidant or lubricant and others. A buffering agent can be but is not limited to sodium di-hydrogen phosphate, potassium di-hydrogen phosphate, Tris-buffer, or any other physiologically and pharmaceutically acceptable buffer which can elevate pH. The functional additive is compatible with the active agent.

In all embodiments of all aspects of the present disclosure, the inert carrier/diluent/disaggregating/deagglomerating agent can be any one of lactose monohydrate, lactose, a lactose functional analogue, or any mixture of at least two thereof. A lactose functional analogue can be but is not limited to dextrose, sorbitol, mannitol, maltitol and xylitol, or a cellulose or cellulose derivative or starch or starch derivative. For example, lactose powder is used as a carrier in nasal drugs and has no effect on drug absorption or the nasal epithelium in short and long term follow up [(10), (11), (12)].

In all aspects and embodiments of the present disclosure the present pharmaceutical composition is substantially free of excipients other than the at least one functional additive comprised in said first type particles and the inert carrier/diluent/disaggregating/deagglomerating agent comprised in said second type particles.

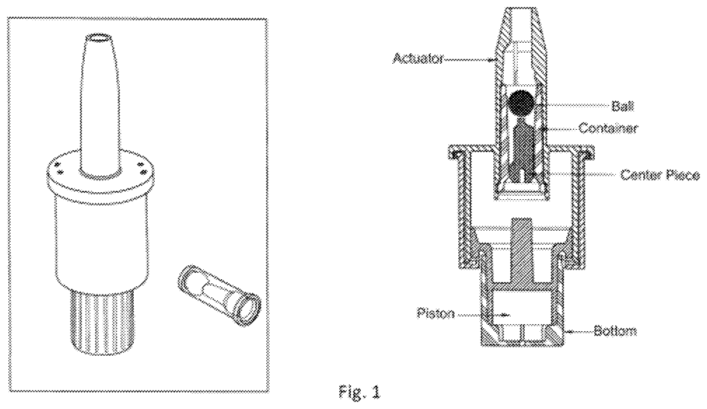

The pharmaceutical composition according to the present disclosure can be contained in disposable dose units for intranasal administration, providing predetermined metered dose of epinephrine. An example of such dose unit is illustrated in FIG. 1, which shows Unitdose Powder Device (UDS), manufactured by Aptar Pharma. Devices of this type for powder spraying are user friendly and designed to enable systemic delivery of small and accurately metered doses of drug formulations by patients or caregivers who are not healthcare professionals or medically trained.

Thus, in a further aspect the present disclosure relates to a dose unit form, specifically a disposable dose unit form, for intranasal administration to a subject of a pharmaceutical composition according to all aspects and embodiments of the present disclosure, wherein the dose unit is loaded with a predetermined dose of the composition and provides the subject with a metered dose the pharmaceutically active ingredient comprised in the composition. As shown in the following Examples, the dose unit device loaded with epinephrine-buffer combination exhibits good product stability under normal and accelerated storage conditions.

Bi-dose and multi-dose intranasal administration devices can be used. Such powder delivery devices generally have a disposable drug containing member and a reusable device body that can be packaged along with a number of drug-containing members. The disposable drug containing member contains the powdered drug within standard size inhalation capsules. Each capsule content equals to one dose.

In addition, syringe-driven and pump-driven spraying atomizers used for delivery of a variety of nasal medications can be used for the delivery of the present pharmaceutical composition.

In specific embodiments of all aspects of the present disclosure, the pharmaceutically active ingredient is epinephrine.

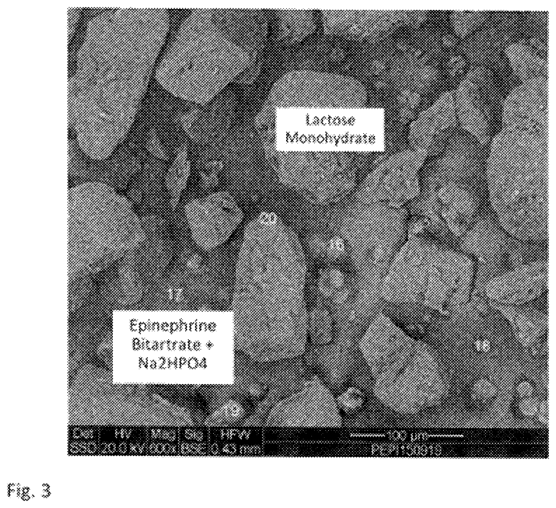

One specific formulation of epinephrine microspheres powder for intranasal administration disclosed herein is also referred to as FMXIN002. Generally, this formulation comprises solid essentially spherical particles of epinephrine bitartrate as the pharmaceutically active ingredient and sodium di-hydrogen phosphate as the pH-adjusting functional additive (first type of particles) and solid irregularly shaped particles of lactose monohydrate as a carrier/diluent/disaggregating/deagglomerating agent (second type of particles). Surprisingly, as shown in FIG. 3 and Example 2, the first type smaller particles contained not only the epinephrine, but also the sodium di-hydrogen phosphate, which was unexpected and is of major advantage reducing any effects of local irritation by epinephrine, as herein discussed. FMXIN002 can be administered intranasally by intranasal delivery devices, for example a disposable intranasal device as described above.

In more detail, FMXIN002 epinephrine microspheres powder is composed of two populations of particles: most of the epinephrine bitartrate and buffer (pH adjusting agent) particles (drug particles), namely at least about 80%, 85%, 90% of the particles or more, have an optimal mean diameter of 10-30 μm, and less than about 10%, 9%, 8%, 7%, 6% or 5% of drug particles have a mean diameter of less than about 5 μm, preventing lung inhalation. The lactose monohydrate particles are larger, and ranges between about 50 to about 200 μm. An in vitro study has demonstrated that immediately after deposition of the powder in the nasal cavity, epinephrine dissolves in the nasal fluid and is rapidly absorbed through the nasal epithelia due to the spherical and small particle size, and high-water solubility (Error! Bookmark not defined.). In specific embodiments, the molar ratio between the epinephrine bitartrate to sodium dihydrogen phosphate is 0.9:1 (weight ratio of 1.67/1).

As mentioned, the first type particles comprise both the active drug and the functional additive, for example epinephrine or salt thereof and a buffering agent such as sodium di-hydrogen phosphate. In other embodiments, buffer materials can be added to epinephrine microsphere to provide pharmaceutical acceptable pH. Some epinephrine salts such as epinephrine bitartrate have acidic pH below 3.5. As mentioned, the administration of microspheres comprising only these salts can cause irritation and discomfort to nasal mucosa. In such cases adjusting the pH in the nasal cavity is recommended, for example by the addition of pH elevating buffers, as described and exemplified herein.

The pH-adjusting agent (buffering agent), for example small amounts of physiological phosphate buffer (sodium di-hydrogen phosphate), provides for maintaining an adequate pH of the epinephrine solution after its dissolution in the nasal mucus, and prevent local irritation or any other discomfort to the patient.

The disclosed pharmaceutical composition can be prepared by a modified spray drying method, as described for example in WO2019/038756. An apparatus for the preparation of the disclosed pharmaceutical composition in the dry powder form essentially comprises the following components:
a) A spray-drying chamber capable of spray-drying a clear and homogeneous solution of the active agent and the functional additive to obtain dry powder particles of said first type, specifically wherein said solution is free of other excipients;
b) A cyclone separator capable of receiving the dry powder particles and the moist air stream from the spray-drying chamber, separating said particles from the moist air through vortex separation, exhausting the air and transferring the separated particles to a receiving chamber through a bag filter; and
c) A receiving chamber pre-filled with a carrier/diluent/disaggregating/deagglomerating agent and adapted for receiving the separated dry powder particles from the cyclone separator, stirring and homogenising said particles with the carrier/diluent/disaggregating/deagglomerating agent to obtain the presently disclosed pharmaceutical composition in dry powder form. The carrier/diluent/disaggregating/deagglomerating agent is capable of colliding and continuously in-situ blending with the particles during the stirring in the receiving chamber, thereby preventing their aggregation and preserving their original size and shape.

The spray-drying chamber is equipped with nozzles, used to produce droplets of the active agent solution, to control the droplet and powder particle size and to maximise heat transfer and the rate of solvent vaporisation. The droplet size may range from 20 to 180 μm, depending on a particular nozzle used. In the present embodiments, the sprayed solution of the active agent is free of any carrier/diluent/disaggregating/deagglomerating agent. The nozzles are designed to spray the solution of the active agent into a hot air flow, thereby achieving a thorough mixing and uniform distribution of the hot air flow and sprayed solution in the spray-drying chamber to allow for substantially complete evaporation of liquids and drying of solid particles of the active agent from the mixture throughout said chamber.

At the laboratory scale, the stirring and homogenisation is achieved by using a magnetic stirrer and a magnetic bar of appropriate size, in addition to the rotation of the receiving chamber. At industrial scale, the stirring and homogenisation may be achieved by using a mechanical stirrer of appropriate size and form, or moving, rotation and vibration of the whole receiving chamber. A conventional spray-drying apparatus contains the empty receiving chamber collecting the dry powder particles of an active agent. This receiver is emptied from time to time in order to ensure the continuous process. In contrast, the present application discloses the receiving chamber pre-filled with a continuously stirred carrier/diluent/disaggregating/deagglomerating agent for preventing aggregation of the dry powder particles and preserving their original size and shape.

Generally, the preparation of presently disclosed pharmaceutical composition by use of the described apparatus comprises the following steps:
A. Preparing a clear and homogeneous solution of at least epinephrine bitartrate or other pharmaceutically acceptable salt thereof or another active epinephrine analogue and a pH-adjusting agent (buffering agent, for example sodium di-hydrogen phosphate) in an organic solvent (for example acetone) or solvent mixture, in a solvent-water or water-miscible solvent mixture, or in water.
B. Filling the receiving chamber with a carrier/diluent/disaggregating/deagglomerating agent and continuously stirring the carrier/diluent/disaggregating/deagglomerating agent in the receiving chamber;
C. Streaming the solution prepared in step (A) together with hot air or gas to the spray-draying chamber, spray-drying the solution in the spray-drying chamber to obtain dry powder particles of said at least one active agent in a moist air or gas, and transferring the obtained dry powder particles and the moist air or gas stream to the cyclone separator;
D. Separating the particles from the moist air or gas through vortex separation in the cyclone separator, exhausting the air or gas and transferring the separated particles to the receiving chamber through a bag filter;
E. Stirring and homogenising said particles obtained in step (D) with the carrier/diluent/disaggregating/deagglomerating agent in the receiving chamber to obtain the presently disclosed pharmaceutical composition in dry powder form; wherein said carrier/diluent/disaggregating/deagglomerating agent is capable of colliding and continuously in-situ blending with the particles during the stirring in the receiving chamber, thereby preventing their aggregation and preserving their original size and shape; and
F. Optionally, additional mixing of the pharmaceutical composition obtained in (E) with an additional amount of the carrier/diluent/disaggregating/deagglomerating agent to achieve the desired active agent-to-carrier ratio in said pharmaceutical composition.

In a further aspect, the present disclosure provides method for treating and/or alleviating a medical condition responsive to an adrenergic receptor agonist, as defined herein, for example by not limited to epinephrine and pharmaceutically acceptable salt thereof. The method of treatment according to the present invention comprises intranasal administration to a subject in need a therapeutically effective amount of an adrenergic receptor agonist pharmaceutical composition as disclosed herein, optionally where loaded in a dose form unit as disclosed herein. In specific embodiments, the adrenergic receptor agonist is epinephrine, more specifically epinephrine bitartrate, at a therapeutically effective amount of IN dose of from about 1.6 mg to about 3.2 mg. Treatment begins with administration of a single dose. If the patient is not stabilized within few minutes, additional doses can be repeatedly administered within 5-15 minutes, and patient is transferred to hospital for further observation. Patients prone to anaphylactic shock or caregiver should be routinely equipped with 2 device packages. Intranasal administration can be to one or both nostrils, as instructed.

Further provided herein is a kit for the treatment of anaphylaxis. The kit comprises at least one dose unit, preferably two dose units of epinephrine as disclosed herein and instructions for use.

In the following description, various aspects of the present application will be described. For purposes of explanation, specific configurations and details are set forth in order to provide a thorough understanding of the present application. However, it will also be apparent to one skilled in the art that the present application may be practiced without the specific details presented herein. Furthermore, well-known features may be omitted or simplified in order not to obscure the present application.

The terminology used herein is for describing particular embodiments only and is not intended to be limiting of the invention. The term “comprising” and “comprises”, used in the claims, should not be interpreted as being restricted to the components and steps listed thereafter; they do not exclude other components or steps. They need to be interpreted as specifying the presence of the stated features, integers, steps and/or components as referred to, but does not preclude the presence and/or addition of one or more other features, integers, steps or components, or groups thereof. Thus, the scope of the expression “a composition comprising A and B” should not be limited to compositions consisting only of components A and B. Also, the scope of the expression “a method comprising the steps X and Z” should not be limited to methods consisting exclusively of those steps.

The dry powder pharmaceutical compositions disclosed herein are characterized by physical and chemical stability, maintaining their integrity throughout manufacture, storage, and device actuation. As shown in Example 4, compositions comprising epinephrine remained stable after three months of storage at 40 °C with 75% relative humidity, maintaining at least 80% of initial drug assay, stable water content, and no visible evidence of particle caking or agglomeration. This stability profile, particularly the absence of visible agglomeration or particle coalescence during storage, reflects the effectiveness of the formulation architecture in preventing particle-to-particle contact and coalescence. As shown in Example 10, when the composition of Example 4 is actuated through an intranasal delivery device, the emitted particle size distribution is essentially the same as the bulk distribution. This confirms the stability studies and suggests that the composition may achieve its effect in preserving particle size distribution as a direct, predictable consequence of agglomeration prevention.

This finding represents a significant advance in the field of intranasal delivery, providing compositions in which the particle size distribution is a stable, reproducible, and predictable property across the full lifecycle from manufacture via delivery and through clinical use.

The emitted Dv90 range disclosed herein may reflect physiological considerations related to intranasal drug delivery. The nasal turbinate mucosa is the primary site of systemic absorption following intranasal administration, and is bathed in approximately 0.1 to 0.2 mL of nasal mucosal fluid. For efficient absorption, drug particles dissolve in this available fluid volume and particle dissolution rate is related to surface-area-to-volume ratio. Thus, the emitted D90 may be selected to balance particle size with dissolution efficiency and absorption rate enabling more rapid epinephrine release and absorption. The preservation of the particle size distribution from bulk to emission, as disclosed herein, may be achieved through a specific formulation architecture described herein.

Hence, in accordance with some aspects, the present disclosure provides a dry powder pharmaceutical composition for intranasal administration comprising an anti-anaphylactic adrenergic receptor agonist, or a pharmaceutically acceptable salt thereof, and a pharmaceutically acceptable carrier, wherein the pharmaceutical composition is characterized in that the composition, when emitted from an intranasal powder delivery device, has an emitted particle size distribution with an emitted Dv90 of less than about 400 microns. In some embodiments, the adrenergic receptor agonist is epinephrine, norepinephrine, dopamine, or antihistamine or pharmaceutically acceptable salts or derivatives thereof.

In accordance with some aspects, the present disclosure provides a dry powder pharmaceutical composition for intranasal administration comprising epinephrine or a pharmaceutically acceptable salt thereof and a pharmaceutically acceptable carrier, wherein the pharmaceutical composition is characterized in that the composition, when emitted from an intranasal powder delivery device, has an emitted particle size distribution with an emitted Dv90 of less than about 450 microns (mm).

In accordance with some aspects, the present disclosure provides a dry powder pharmaceutical composition for intranasal administration comprising epinephrine or a pharmaceutically acceptable salt thereof and a pharmaceutically acceptable carrier, wherein the pharmaceutical composition is characterized in that the composition, when emitted from an intranasal powder delivery device, has an emitted particle size distribution with an emitted Dv90 of less than about 400 microns.

It should be noted that the dry powder pharmaceutical composition as disclosed herein is administered intranasally by means of an intranasal powder delivery device configured to spray the dry powder composition into the nasal passage of the subject, thereby providing the emitted particle size distribution as defined herein. Suitable intranasal powder delivery devices include, but are not limited to, unit-dose powder devices, bi-dose and multi-dose powder devices, breath-powered nasal delivery devices, pump-driven powder atomizers, and syringe-driven powder sprayers. In a specific embodiment, the intranasal powder delivery device is a disposable unit-dose powder device, for example the Unitdose Powder Device (UDS) manufactured by Aptar Pharma, as illustrated in FIG. 1.

As used herein, the term “emitted particle size distribution” or “ePSD” refers to the particle size distribution of the dry powder pharmaceutical composition at the point of nasal delivery. This nature of the emitted particle size distribution reflects the physical and chemical stability of the composition. The emitted particle size distribution is thus a characteristic property of the composition itself, reproducible across different intranasal delivery devices and actuation conditions, and is not dependent on device-mediated mechanisms such as agglomeration, deagglomeration, or air-assisted particle size modification.

The emitted particle size distribution may be determined by actuating the intranasal powder delivery device into a stirred non-aqueous medium, for example Isopar G stirred at about 2000 rpm, and measuring the resulting particle size distribution using laser diffraction.

In some examples, the composition has an emitted particle size distribution with an emitted Dv90 of less than about 350 microns. In some examples, the composition has an emitted particle size distribution with an emitted Dv90 of less than about 300 microns. In some examples, the composition has an emitted particle size distribution with an emitted Dv90 of less than about 250 microns. In some examples, the composition has an emitted particle size distribution with an emitted Dv90 of less than about 200 microns. In some examples, the composition has an emitted particle size distribution with an emitted Dv90 of less than about 150 microns.

In some embodiments, the composition has an emitted particle size distribution with an emitted Dv90 of between about 100 μm and about 450 μm. In some embodiments, the composition has an emitted particle size distribution with an emitted Dv90 of between about 100 μm and about 400 μm. In some embodiments, the composition has an emitted particle size distribution with an emitted Dv90 of between about 100 μm and about 350 μm. In some embodiments, the composition has an emitted particle size distribution with an emitted Dv90 of between about 100 μm and about 300 μm. In some embodiments, the composition has an emitted particle size distribution with an emitted Dv90 of between about 100 μm and about 250 μm. In some embodiments, the composition has an emitted particle size distribution with an emitted Dv90 of between about 100 μm and about 200 μm. In some embodiments, the composition has an emitted particle size distribution with an emitted Dv90 of between about 100 μm and about 180 μm. In some embodiments, the composition has an emitted particle size distribution with an emitted Dv90 of between about 100 μm and about 160 μm.

In accordance with some aspects, the present disclosure provides a dry powder pharmaceutical composition for intranasal administration comprising an anti-anaphylactic adrenergic receptor agonist, or a pharmaceutically acceptable salt thereof, and a pharmaceutically acceptable carrier, wherein the pharmaceutical composition is characterized in that the composition, when emitted from an intranasal powder delivery device, has an emitted particle size distribution with an emitted Dv50 between about 25 microns and about 200 microns. In some embodiments, the adrenergic receptor agonist is epinephrine, norepinephrine, dopamine, or antihistamine or pharmaceutically acceptable salts or derivatives thereof.

In some aspects, which may be considered as embodiments of the invention, the dry powder pharmaceutical composition for intranasal administration, comprising epinephrine or a pharmaceutically acceptable salt thereof and a pharmaceutically acceptable carrier is characterized in that the composition, when emitted from an intranasal powder delivery device, has an emitted particle size distribution with an emitted Dv50 between about 25 microns and about 200 microns.

In some aspects, which may be considered as embodiments of the invention, the dry powder pharmaceutical composition is formulated to provide, upon intranasal administration, an emitted particle size distribution characterized by an emitted D50 of about 25 μm to about 200 μm.

In some examples, the composition has an emitted particle size distribution with an emitted Dv50 between about 25 microns and about 200 microns. The emitted D50 of about 25 μm to about 200 μm may reflect a particle size range suitable for deposition on the nasal turbinate mucosa.

In some examples, the composition has an emitted particle size distribution with an emitted Dv50 between about 30 microns and about 200 microns. In some examples, the composition has an emitted particle size distribution with an emitted Dv50 between about 40 microns and about 200 microns. In some examples, the composition has an emitted particle size distribution with an emitted Dv50 between about 50 microns and about 200 microns. In some examples, the composition has an emitted particle size distribution with an emitted Dv50 between about 60 microns and about 200 microns.

In some examples, the composition has an emitted particle size distribution with an emitted Dv50 between about 25 microns and about 180 microns. In some examples, the composition has an emitted particle size distribution with an emitted Dv50 between about 25 microns and about 150 microns. In some examples, the composition has an emitted particle size distribution with an emitted Dv50 between about 25 microns and about 120 microns. In some examples, the composition has an emitted particle size distribution with an emitted Dv50 between about 25 microns and about 100 microns.

In some examples, the composition has an emitted particle size distribution with an emitted Dv50 between about 30 microns and about 180 microns. In some examples, the composition has an emitted particle size distribution with an emitted Dv50 between about 40 microns and about 150 microns. In some examples, the composition has an emitted particle size distribution with an emitted Dv50 between about 50 microns and about 100 microns.

In some aspects which may be considered as embodiments of the invention, the pharmaceutical composition is characterized in that the composition, when emitted from an intranasal powder delivery device, has an emitted particle size distribution with an emitted Dv10 between about 5 microns and about 25 microns.

In some examples, the composition has an emitted particle size distribution with an emitted Dv10 between about 5 microns and about 25 microns.

In some examples, the composition has an emitted particle size distribution with an emitted Dv10 between about 5 microns and about 20 microns. In some examples, the composition has an emitted particle size distribution with an emitted Dv10 between about 5 microns and about 15 microns. In some examples, the composition has an emitted particle size distribution with an emitted Dv10 between about 5 microns and about 10 microns.

In some aspects which may be considered as embodiments of the invention, the pharmaceutical composition is characterized in that the composition, when emitted from an intranasal powder delivery device, has an emitted particle size distribution with an emitted Dv50 between about 25 microns and about 200 microns and an emitted Dv10 between about 5 microns and about 25 microns.

The presently disclosed compositions exhibit an emitted particle size distribution that is essentially the same as the bulk distribution. This “emitted≈bulk” behavior is a direct consequence of the formulation architecture. As demonstrated in Example 4, the bulk particle size distribution maintains stability throughout storage, with no visible agglomeration or caking.

As used herein, the term “bulk particle size distribution” or “bPSD” refers to the particle size distribution of the dry powder pharmaceutical composition as loaded for intranasal administration, prior to delivery to the nasal passage. The bPSD reflects the combined volume-weighted size distribution of all particle populations in the bulk powder, including both the first type of solid particles comprising the active agent and the second type of solid particles comprising the carrier as described herein.

The relationship between the bulk particle size distribution and the emitted particle size distribution of the presently disclosed compositions is illustrated in Table 3. As shown in this table, the emitted particle size distribution is essentially the same as the bulk particle size distribution. Specifically, the emitted D50 (69.2 μm) is essentially the same as the bulk D50 (81.4 μm), the emitted D10 (8.7 μm) is with is essentially the same as the bulk D10 (8.3 μm), and the emitted D90 (127 μm) is essentially the same as the bulk D90 (151 μm). These results demonstrate that the presently disclosed compositions substantially preserve the particle size distribution through actuation of the intranasal delivery device.

In some aspects which may be considered as embodiments of the invention, the composition has an emitted particle size distribution that is essentially the same as a bulk particle size distribution. In some aspects which may be considered as embodiments of the invention, the composition when emitted from an intranasal powder delivery device, has an emitted particle size distribution hat is essentially the same as a bulk particle size distribution.

As used herein, the phrase “essentially the same as” when used to describe the relationship between the emitted particle size distribution and the bulk particle size distribution means that the corresponding D10, D50, and D90 values of the emitted particle size distribution each do not differ from the corresponding D10, D50, and D90 values of the bulk particle size distribution by more than about 20%. For example, an emitted D50 that is essentially the same as the bulk D50 means that the emitted D50 is within about 20% (and in some embodiments within about 15%) of the bulk D50 value. Similarly, an emitted D10 that is essentially the same as the bulk D10 means that the emitted D10 is within about 20% of the bulk D10 value, and an emitted D90 that is essentially the same as the bulk D90 means that the emitted D90 is within about 20% of the bulk D90 value. The data of Example 10 (Table 3) demonstrate this relationship empirically: the emitted D50 differs from bulk D50 by 15%, the emitted D10 differs from bulk D10 by 5%, and the emitted D90 differs from bulk D90 by 16%, all well within the 20% tolerance.

In some examples, the bulk particle size distribution and the emitted particle size distribution is characterized by an emitted Dv10 that is essentially the same as bulk Dv10.

In some examples, the bulk particle size distribution and the emitted particle size distribution is characterized by an emitted Dv50 that is essentially the same as bulk Dv50.

In some examples, the bulk particle size distribution and the emitted particle size distribution is characterized by an emitted Dv90 that is essentially the same as bulk Dv90.

In some examples, the bulk particle size distribution and the emitted particle size distribution is characterized by an emitted Dv10 that is essentially the same as bulk Dv10, an emitted Dv50 that is essentially the same as bulk Dv50 and an emitted Dv90 that is essentially the same as bulk Dv90.

In some examples, the bulk particle size distribution is characterized by a bulk Dv10 of about 5 μm to about 25 μm. In some examples, the bulk particle size distribution is characterized by a bulk Dv10 of between about 5 μm and about 25 μm. In some examples, the bulk particle size distribution is characterized by a bulk Dv10 of between about 5 μm and about 20 μm. In some examples, the bulk particle size distribution is characterized by a bulk Dv10 of between about 5 μm and about 15 μm. In some examples, the bulk particle size distribution is characterized by a bulk Dv10 of between about 5 μm and about 10 μm.

In some examples, the bulk particle size distribution is characterized by a bulk Dv50 of about 25 μm to about 200 μm. In some examples, the bulk particle size distribution is characterized by a bulk Dv50 of between about 50 μm and about 150 μm. In some examples, the bulk particle size distribution is characterized by a bulk Dv50 of between about 60 μm and about 150 μm. In some examples, the bulk particle size distribution is characterized by a bulk Dv50 of between about 70 μm and about 150 μm. In some examples, the bulk particle size distribution is characterized by a bulk Dv50 of between about 80 μm and about 150 μm.

In some examples, the bulk particle size distribution is characterized by a bulk Dv50 of between about 50 μm and about 140 μm. In some examples, the bulk particle size distribution is characterized by a bulk Dv50 of between about 50 μm and about 130 μm. In some examples, the bulk particle size distribution is characterized by a bulk Dv50 of between about 50 μm and about 120 μm. In some examples, the bulk particle size distribution is characterized by a bulk Dv50 of between about 50 μm and about 100 μm.

In some examples, the bulk particle size distribution is characterized by a bulk Dv90 of less than about 400 μm. In some examples, the bulk particle size distribution is characterized by a bulk Dv90 of between about 100 μm and about 450 μm. In some examples, the bulk particle size distribution is characterized by a bulk Dv90 of between about 100 μm and about 400 μm. In some examples, the bulk particle size distribution is characterized by a bulk Dv90 of between about 100 μm and about 350 μm. In some examples, the bulk particle size distribution is characterized by a bulk Dv90 of between about 100 μm and about 300 μm. In some examples, the bulk particle size distribution is characterized by a bulk Dv90 of between about 100 μm and about 250 μm. In some examples, the bulk particle size distribution is characterized by a bulk Dv90 of between about 100 μm and about 200 μm. In some examples, the bulk particle size distribution is characterized by a bulk Dv90 of between about 100 μm and about 180 μm. In some examples, the bulk particle size distribution is characterized by a bulk Dv90 of between about 100 μm and about 160 μm.

As described herein, the dry powder pharmaceutical composition comprises a first type of solid particles comprising an anti-anaphylactic adrenergic receptor agonist, for example, epinephrine or a pharmaceutically acceptable salt thereof and a physiologically acceptable buffering agent, and a second type of solid particles comprising a pharmaceutically acceptable carrier. As used herein, the “first type of solid particles” refers to the active agent-containing particles produced by spray-drying of a solution comprising the active agent and the buffering agent, resulting in essentially spherical particles in which the active agent and buffering agent are present as a homogeneous combination. As used herein, the “second type of solid particles” refers to the carrier particles, which are substantially irregularly shaped particles of the pharmaceutically acceptable carrier.

In some aspects, the dry powder pharmaceutical composition comprising the first type and second type of solid particles as defined herein is formulated to provide, upon intranasal administration, an emitted particle size distribution that is essentially the same as the bulk particle size distribution of the dry powder pharmaceutical composition prior to emission. Without being bound by theory, the essentially same bulk and emitted particle size distributions arise from the two-population formulation architecture, in which the second type of solid particles, the carrier particles, acts to maintain the separation and dispersion of the first type of solid particles, the active agent particles, during storage in the device reservoir and during actuation, thereby preventing agglomeration of the active agent particles and preserving the particle size distribution through emission.

In some examples, the composition comprises: a first type of solid particles comprising an anti-anaphylactic adrenergic receptor agonist or a pharmaceutically acceptable salt thereof and a physiologically acceptable buffering agent; and a second type of solid particles comprising a pharmaceutically acceptable carrier.

In some examples, the composition comprises: a first type of solid particles comprising epinephrine or a pharmaceutically acceptable salt thereof and a physiologically acceptable buffering agent; and a second type of solid particles comprising a pharmaceutically acceptable carrier.

In some examples, at least about 90% of the first type of solid particles have a mean particle size of about 10 μm to about 40 μm, and wherein the second type of solid particles have a mean particle size of about 50 μm to about 200 μm. In some embodiments, at least about 90%, 92%, 95%, or 98% of the first type of solid particles have a mean particle size of about 10 μm to about 30 μm. In some embodiments, less than about 10%, less than about 8%, less than about 6%, or less than about 5% of the first type of solid particles have a mean particle size of less than about 5 μm

The size differential between the first type of solid particles (about 10 μm to about 40 μm) and the second type of solid particles (about 50 μm to about 200 μm) may enable the carrier particles to act as a physical barrier preventing agglomeration of the active agent particles, and contributes to the preservation of the particle size distribution upon emission from the device.

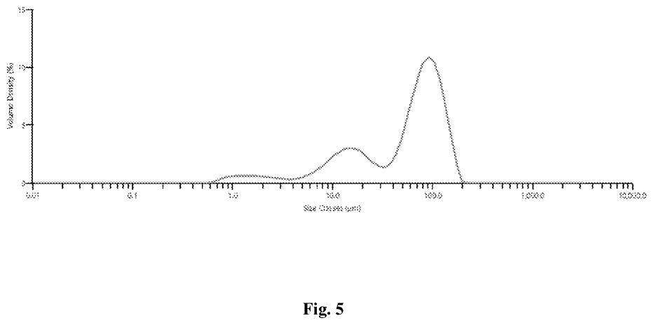

The two-population formulation architecture of the presently disclosed compositions gives rise to a bimodal particle size distribution, both in the bulk powder and in the emitted powder. The bimodal nature of the distribution reflects the presence of two distinct particle populations. It is noted that the D10, D50, and D90 parameters of the bulk and emitted particle size distributions reflect the combined volume-weighted distribution of both particle populations and are not independently indicative of the mean particle sizes of the individual first and second type particle populations. As shown in FIG. 5, the bimodal particle size distribution of the emitted powder is substantially preserved relative to the bulk powder, confirming that both particle populations remain intact following actuation of the intranasal delivery device. The first population of buffered epinephrine particles, visible as the smaller mode at about 10 μm to about 40 μm in FIG. 5, and the second population of lactose monohydrate carrier particles, visible as the larger mode at about 50 μm to about 200 μm in FIG. 5, are both present in the emitted powder without significant agglomeration or disruption.

In some embodiments, the composition comprises two distinct particle populations after emission from the intranasal powder delivery device. As used herein, “two distinct particle populations” refers to a first population comprising the an anti-anaphylactic adrenergic receptor agonist, for example, epinephrine particles and a second population comprising the carrier particles, wherein both populations are preserved in the emitted powder and are identifiable as distinct populations in the bimodal particle size distribution of the emitted powder, as illustrated in FIG. 5.

In some examples, the first type of solid particles comprises epinephrine bitartrate and sodium dihydrogen phosphate, and wherein the second type of solid particles comprises lactose monohydrate. The combination of epinephrine bitartrate and sodium dihydrogen phosphate in the first type of solid particles provides a co-spray-dried buffered active agent particle in which the pH of the dissolved epinephrine solution following dissolution in nasal mucosal fluid is maintained within a physiologically acceptable range, reducing local irritation and discomfort at the nasal mucosa. In a specific embodiment, the molar ratio of epinephrine bitartrate to sodium dihydrogen phosphate in the first type of solid particles is about 0.9:1. In some embodiments, the second type of solid particles comprises lactose monohydrate. The lactose monohydrate carrier particles are substantially irregularly shaped, having a mean particle size of about 50 μm to about 200 μm, and may serve as the disaggregating and deagglomerating agent that maintains the dispersion of the first type of solid particles in the formulation.

As disclosed herein, the two-population formulation architecture achieves sustained prevention of agglomeration both during storage and during device actuation. The large carrier particles (approximately 50-200 μm) maintain their role as physical spacers throughout the composition's shelf life, preventing coalescence of smaller active agent particles even under accelerated storage conditions (e.g., 40 °C/75% RH for three months).

In accordance with some aspects, which may be considered as embodiments of the invention, the dry powder pharmaceutical composition for intranasal administration comprises epinephrine or a pharmaceutically acceptable salt thereof and a pharmaceutically acceptable carrier, and is characterized in that the composition is substantially free of agglomerated particles and retains at least 80% of initial drug assay after storage for 3 months at 40 °C with 75% relative humidity. As shown herein, the composition maintains agglomeration prevention throughout the composition's shelf life, preserving both physical and chemical stability.

The compositions are formulated with a therapeutically effective dose of epinephrine for intranasal delivery. In some examples, a single dose of the dry powder pharmaceutical composition comprises between about 1.6 mg and about 5.5 mg of epinephrine or the pharmaceutically acceptable salt thereof. In some embodiments, the single dose of about 1.6 mg to about 5.5 mg is contained in a dry powder fill weight of about 17.5 mg to about 65 mg per device, such that the epinephrine loading in the formulation is about 4% to about 10% w/w of the total powder fill weight

In some examples, a single dose comprises between about 1.6 mg and about 4.8 mg. In some examples, a single dose comprises between about 1.6 mg and about 4.5 mg. In some examples, a single dose comprises between about 3.2 mg and about 5.5 mg. In some examples, a single dose comprises between about 4.0 mg and about 5.5 mg. In some specific examples, the single dose comprises about 1.6 mg, about 3.2 mg, about 3.6 mg, about 4.0 mg, about 4.5 mg, about 4.8 mg, or about 5.5 mg of epinephrine or the pharmaceutically acceptable salt thereof.

In some aspects, the dry powder pharmaceutical composition disclosed herein is provided in a disposable dose unit form for intranasal administration to a subject. As used herein, “disposable dose unit form” refers to a single-use intranasal powder delivery device pre-loaded with a predetermined single dose of the dry powder pharmaceutical composition, configured to emit the dose upon a single actuation and intended for disposal following use. The disposable dose unit form is loaded with a predetermined single dose of the dry powder pharmaceutical composition and is configured, upon actuation, to emit the dry powder pharmaceutical composition having an emitted particle size distribution characterized by an emitted D50 of about 25 μm to about 200 μm.

An example of a suitable disposable dose unit form is the Unitdose Powder Device (UDS) manufactured by Aptar Pharma, as illustrated in FIG. 1. The Aptar UDS device is a user-friendly single-use intranasal powder delivery device designed to enable accurate and metered delivery of dry powder pharmaceutical compositions by patients or caregivers without medical training. Upon actuation, the device produces an airflow through the powder reservoir that delivers the predetermined dose of the dry powder composition into the nasal passage of the subject.

A key feature of the disposable dose unit form in combination with the dry powder pharmaceutical compositions disclosed herein is that the emitted particle size distribution produced upon actuation is essentially the same as the bulk particle size distribution of the composition loaded into the device prior to actuation, as set forth in Table 3. This device-independent PSD behaviour ensures that the therapeutic dose of epinephrine is delivered with a consistent and predictable particle size distribution to the nasal turbinate mucosa, regardless of variations in actuation technique, device orientation, or user-specific factors.

The dry powder pharmaceutical compositions and disposable dose unit forms disclosed herein are intended for use in the treatment of medical conditions requiring rapid systemic delivery of epinephrine.

Hence, in some aspects the present disclosure provides a method of treating anaphylaxis or anaphylactic shock in a subject in need thereof, comprising intranasally administering to the subject a therapeutically effective amount of the dry powder pharmaceutical composition as disclosed herein. As used herein, “anaphylaxis” refers to a severe, life-threatening systemic allergic reaction characterized by rapid onset and involving multiple organ systems, including the cardiovascular, respiratory, and cutaneous systems. As used herein, “anaphylactic shock” refers to the hemodynamic manifestation of anaphylaxis, characterized by a critical decrease in blood pressure due to vasodilation and increased vascular permeability.

In some embodiments, the method of treating anaphylaxis or anaphylactic shock comprises administering a single dose of about 1.6 mg to about 5.5 mg of epinephrine or the pharmaceutically acceptable salt thereof intranasally. In some embodiments, intranasal administration is to one or both nostrils of the subject.

In some aspects, the present disclosure provides a method of intranasally delivering epinephrine to a subject in need thereof, comprising actuating an intranasal powder delivery device containing the dry powder pharmaceutical composition as disclosed herein, wherein actuation of the intranasal powder delivery device emits the dry powder pharmaceutical composition having an emitted particle size distribution characterized by an emitted D50 of about 25 μm to about 200 μm. As used herein, “intranasally delivering epinephrine” encompasses delivery for any therapeutic indication responsive to epinephrine administration, including but not limited to anaphylaxis, anaphylactic shock, bronchospasm, cardiac arrest, and other conditions in which rapid systemic epinephrine delivery is indicated.

In some embodiments, the method of intranasally delivering epinephrine comprises placing the delivery head of the intranasal powder delivery device into a nasal passage of the subject and actuating the device to produce an airflow through the reservoir, thereby emitting the dry powder pharmaceutical composition into the nasal passage. In some embodiments, actuation comprises manually compressing the device to produce an airflow pressure sufficient to deliver the dose into the nasal passage. In some embodiments, the emitted particle size distribution produced upon actuation is essentially the same as the bulk particle size distribution of the dry powder pharmaceutical composition prior to actuation, as described herein, ensuring consistent delivery of epinephrine to the nasal turbinate mucosa independently of user technique or device orientation.

### DEFINITIONS

The terms “drug”, “active substance”, “API” (Active Pharmaceutical Ingredient) or “active principle” or “active ingredient”, “pharmaceutically active agent”, “pharmaceutically active ingredient”, “active substance”, “active molecule”, “active compound” and the like used herein interchangeably, refer to a pharmaceutically active substance that provides a therapeutic/physiological effect to a patient, and can also refer to a mixture of at least two thereof.

The terms “formulation”, “pharmaceutical formulation”, “composition” and “pharmaceutical composition” may be used herein interchangeably, and are to be taken to mean a formulation comprising an adrenergic receptor agonist, such as but not limited to epinephrine or a pharmaceutically active salt thereof for use in therapy/medicine.

The terms “inert” or “inactive” or “inactive ingredient” or “inert ingredient”, as used interchangeably herein refer to components of the pharmaceutical composition, or used in the preparation thereof, that do not instantly react with the active ingredient or adversely affect its properties, or cause any biological effect upon administration to a subject when administered at reasonable amounts to a subject. The general examples of these components are described in “The Handbook of Pharmaceutical Excipients”, 4th Edition, by Rowe, Sheskey and Weller, Pharmaceutical press, 2003. Additional exemplary list is Inactive Ingredients Guide of the Food and Drug Administration, USA.

“Carrier”, “diluent”, “disaggregating agent” and “deagglomerating agent” are used herein interchangeably, and refer to an inert ingredient added to the pharmaceutical composition.

A “patient” or “subject” that may be administered with the pharmaceutical composition and/or dose units loaded therewith according to the presently disclosed subject matter. In general, where the drug is an adrenergic receptor agonist as herein described, the “patient” or “subject” is a human, suffering from a medical condition responsive to such agonist. Such conditions may be cardiac arrest and other heart problems, patients prone to anaphylactic shock including all Type 1 allergy patients, asthmatic patients and others.

“An adrenergic receptor agonist” as used herein is to be taken to mean an agent that stimulates a response from adrenergic receptors. An examples of such agonists are epinephrine (adrenaline) and its pharmaceutically acceptable salts. “Epinephrine” as used herein also refers to pharmaceutically active salts thereof.

“pH adjusting agent”, “buffering agent” and “buffer” as used herein interchangeably are to be taken to mean any chemical agent that affects the pH of its immediate environment.

The term a composition or substance “substantially free of excipients” is to be taken to mean that it does contain more than 5% of such excipient/s.

The terms “treat”, or forms thereof, and the term “alleviate” and the like are to be taken to mean at least partially ameliorate or cure or totally eliminate the patient's condition as defined herein.

The term “intranasal administration” as used herein is to be taken to mean nasal application in one or both nostrils of the subject.

The term “suitable” as used herein is to be taken to mean having the properties that enable providing the defined result.

“About” as used herein generally refers to approximate values. When referred to a dose of drug, or size of particles and the like, “about” should be understood as including the range of a value ±15%. When referred to other values, the term should be understood as including the range of a value ±15%, for example ±15%, ±12%, ±10%, ±8%, ±5%, ±2% or ±1%. Other similar terms, such as “substantially”, “generally”, “up to” and the like are to be construed as modifying a term or value such that it is not an absolute. Such terms will be defined by the circumstances and the terms that they modify as those terms are understood by those of skilled in the art. This includes, at very least, the degree of expected experimental error, technical error and instrumental error for a given experiment, technique or an instrument used to measure a value.

As used herein, the term “and/or” includes any combinations of one or more of the associated listed items. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealised or overly formal sense unless expressly so defined herein. Well-known functions or constructions may not be described in detail for brevity and/or clarity.

As used in the specification and claims, the forms “a”, “an” and “the” include singular as well as plural references unless the context clearly dictates.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The presently disclosed subject matter is further illustrated by the following examples, which are illustrative only and are not to be construed as limiting the scope of the invention. Variations and equivalents of these examples will be apparent to those skilled in the art in light of the present disclosure, the drawings and the claims herein.

It is appreciated that certain features of the presently disclosed subject matter which are, for clarity, described in the context of separate embodiments, can also be provided in combination in a single embodiment. Conversely, various features of the presently disclosed subject matter, which are, for brevity, described in the context of a single embodiment, can also be provided separately or in any suitable sub-combination.

Although the presently disclosed subject matter has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent and patent application was specifically and individually indicated to be incorporated herein by reference. In addition, citation or identification of any reference in this application shall not be construed as an admission that such reference is available as relevant prior art to the presently disclosed subject matter.

### DESCRIPTION OF NON-LIMITING EXAMPLES

### Materials

Epinephrine bitartrate (TransoPharma); Sodium Phosphate Dibasic Dihydrate (Merck), lactose monohydrate (Meggle Pharma); acetone (BioLab).

### Methods

The spray-drying process was carried out using the Mini Spray Dryer B-290 of Büchi Labortechnik AG. A magnetic stirrer (Fried Electric) was placed under the receiver (receiving chamber), a magnetic bar of appropriate size was inserted into the receiver, and then the carrier/diluent/disaggregating/deagglomerating agent was added. The liquid feed containing at least one active agent was prepared by dissolving at least one active compound in the selected solvent or mixture of solvents. Quantification was performed using HPLC and a Dionex HPLC instrument. A FEI Quanta-200 Scanning Electron Microscope (SEM) equipped with an Everhart-Thornley Detector was used to obtain the images of the spray-dried powder. The accelerating voltage of 20 kV was applied to provide magnification from 250 to 10,000 times. In addition, an X-ray Element Analysis Detector (Link ISIS, Oxford Instruments, England) was used to determine particles morphology and chemical composition and their distribution throughout Dry Powder Inhaler (DPI). Particle size was measured using the Malvern Mastersizer 3000 series based on the Light Diffraction method.

Epinephrine assay in the compositions was determined using Dionex HPLC-PDA instrument equipped with Chromeleon software; Column & packing: Thermo ODS, 3 μu 100×4.6 mm Cat No: 30103-104630 or equivalent
Mobile phase A: Buffer: Acetonitrile (95:5, v/v)
Mobile phase B: Buffer: Acetonitrile (55:45, v/v)
Flow rate: 1.2 mL/min

### Gradient Table for Sample:

| Time, min | % A | % B |
|---|---|---|
| 0.0 | 95 | 5 |
| 20 | 50 | 50 |
| 21 | 50 | 50 |
| 23 | 95 | 5 |
| 30 | 95 | 5 |

### Gradient Table for Standard:

| Time, min | % A | % B |
|---|---|---|
| 0.0 | 95 | 5 |
| 10 | 72.5 | 27.5 |
| 10.5 | 95 | 5 |
| 15 | 95 | 5 |

Injection volume: 20 μL
Detector PDA: UV, 210 nm; 200-400 nm for identification.
Column temperature: 50 °C.
Auto sampler temperature: ambient
Run time 30 min
Diluent: Mobile Phase A
RT of epinephrine peak: 5-7 min

### Example 1: Modification of the Commercial Büchi Labortechnik AG Spray-Dryer

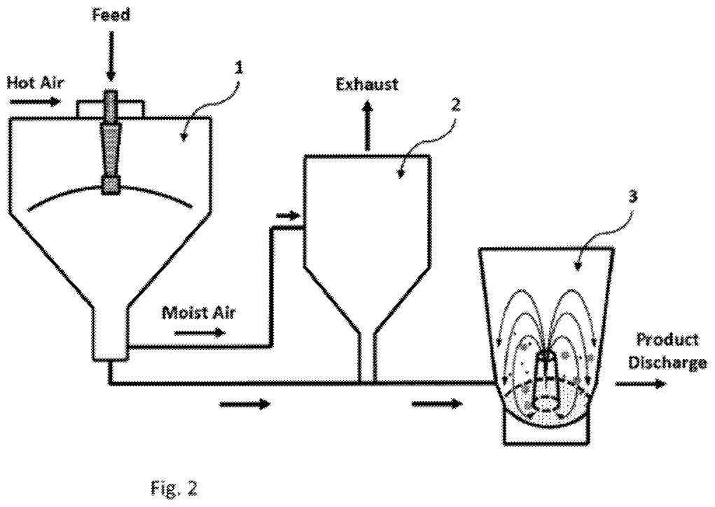

FIG. 2 schematically shows a modified spray dryer used in the present examples. A Mini Spray-Dryer B-290 of Büchi Labortechnik AG was modified by:
1. Addition of a magnetic bar into the glass receiver and placing a magnetic stirrer under the continuously rotating glass receiver of the spray-dryer.
2. Selection of a suitable two-fluids spraying nozzle for spraying the solution containing only the active agent epinephrine (without carrier/diluent/disaggregating/deagglomerating agent) into fine droplets suitable for the preparation of 10-30 μm dry powder particles of the active agent. One of the fluids is the clear and homogeneous solution of the active agent, and the second fluid is the drying gas.

The modified spray dryer is suitable for the particles engineering and prevention of agglomeration according to the present disclosure.

### Example 2: Epinephrine Bitartrate/Sodium Di-Hydrogen Phosphate Composition with Lactose Monohydrate

Epinephrine bitartrate (2.5 g) and sodium di-hydrogen phosphate (1.5 g) were dissolved for 20 min in 15 g of acetone and 20 g water mixture under nitrogen and stirring at 300 rpm. An appropriate size magnetic bar was placed in the receiver and lactose monohydrate (3.0 g) was added thereto, with the stirring rate set at 150 rpm. A clear and homogeneous solution of the drug was obtained, and spray-dried using the modified Büchi Mini Spray-Dryer with inlet nitrogen temperature of 130 °C and outlet temperature of 80 °C, thereby obtaining a dry powder of epinephrine bitartrate/sodium di-hydrogen phosphate, which was further blended in-situ with lactose monohydrate in the receiver. Stirring was maintained in the receiver during the entire process. The loading of epinephrine bitartrate/sodium di-hydrogen phosphate in the composition was about 8.7% w/w.

The SEM images presented in FIG. 3 show that the small spherical particles of epinephrine bitartrate/sodium di-hydrogen phosphate have a narrow size distribution of 5-30 μm, and are dispersed between large irregular particles of lactose ranging between 40 □m to 240 μm.

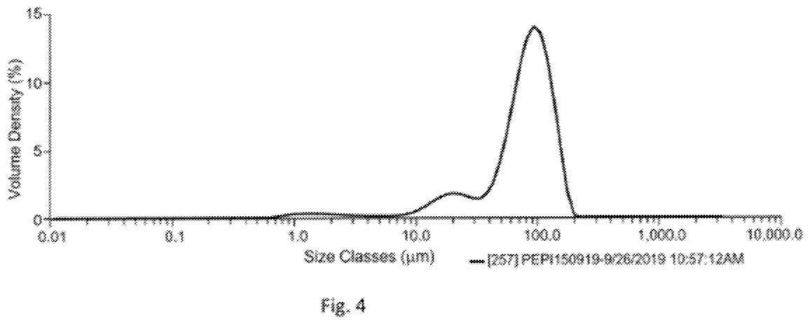

The obtained epinephrine bitartrate/sodium di-hydrogen phosphate composition was subjected to particle size analysis using a Malvern Laser Diffraction instrument. As shown in FIG. 4, the following particle size distribution was obtained: D (10)=21.4 μm, D (50)=82.0 μm and D (90)=133 μm. The amount of the obtained particles having the size less than 10 μm was about 3.7% v/v. 5 μm was about 2.8% v/v. The particle size distribution of epinephrine microspheres thus meets powder Bulk Drug Product specification and safety requirements (11, 13)

### Example 3: Epinephrine Bitartrate Drug-Device Combination Product Preparation

Aptar Unit-Dose Powder disposable devices, assembled according to the manufacturer's guidelines, were filled with the epinephrine bitartrate/sodium di-hydrogen phosphate composition prepared in Example 2. Each device contained 35 mg of powder including 1.6 mg of epinephrine bitartrate.

### Example 4: Stability Data of Epinephrine Drug-Device Combination Product Under Accelerated Aging Conditions

The epinephrine bitartrate combination products prepared in Example 3 were subjected to accelerated aging conditions at 40 °C+2 °C and 75% RH+5% RH. Three months stability data are presented in Table 2.

TABLE 2
Stability data

| | Testing interval, | | Assay | Impurities/related | Water | |
|---|---|---|---|---|---|---|
| Item | months | Appearance | (HPLC) | substances | content | pH |
| Specifications | | Disposable | 1.6 ± 0.32 | A. ≤0.3% | NMT 7% | 5-7 |
| | | plastic device; | mg/device | B. ≤0.2% | | |
| | | white color; | (80.0-120.0%) | C. ≤0.2% | | |
| | | no visible | | D. ≤0.10% | | |
| | | damage | | E. ≤0.10% | | |
| Test result | initial | Disposable | 1.69 mg/device | A. −0.29 | 5.3% | 6.2 |
| | | plastic device; | (105.6%) | B. ≤ND² | | |
| | | white color; | | C. ≤ND | | |
| | | no visible | | D. ≤ND | | |
| | | damage | | E. ≤ND | | |
| | | | | Unspecified | | |
| | | | | impurities: BRL¹ | | |
| | | | | Total Impurities: 0.3% | | |
| Test result | 1 | No change | 1.47 mg/device | A. −0.19% | 5.2% | 6.2 |
| | | | (91.9%) | B. ≤ND | | |
| | | | | C. ≤ BRL¹ | | |
| | | | | D. ≤ND | | |
| | | | | E. ≤ND | | |
| | | | | Unspecified | | |
| | | | | impurities: ND | | |
| | | | | Total Impurities: 0.2% | | |
| Test result | 3 | No change | 1.36 mg/device | A. −0.21% | NA² | NA² |
| | | | (84.7%) | B. ≤ND | | |
| | | | | C. ≤ND | | |
| | | | | D. ≤ND | | |
| | | | | E. ≤ND | | |
| | | | | Unspecified | | |
| | | | | impurities: | | |
| | | | | (RRT 1.4)- | | |
| | | | | 0.05% | | |
| | | | | (RRT 1.6)- | | |
| | | | | 0.07% | | |
| | | | | (RRT 2.9)- | | |
| | | | | 0.09% | | |
| | | | | Total Impurities: 0.42% | | |

¹below reporting limit (0.05%)
²Not Detected

Conclusions: the powdered epinephrine formulation of the present invention showed good stability after 3 months at 40 °C and 75% relative humidity (RH). It contained 0.8% of the total impurities and similar assay of API. All results meet drug device combination products stability specifications.

### Example 5: In Vivo Study of FMXIN002

In order to mimic PK achieved by intramuscular administration (IM) intranasal administration of epinephrine requires a higher dose. The clinical use of IN epinephrine in humans ranges from 1 mg up to 12 mg for different indications and in different formulations, with no serious adverse events (Error! Bookmark not defined.). Prior studies using IN (intranasal) epinephrine showed 5 or 6 mg IN dose as equivalent to IM injection of 0.3 mg.

Based on clinical evidence, a starting IN dose of 1.6 mg is estimated to be equivalent to an IM dose of 0.1 mg. Hence a starting dose of IN epinephrine 1.6 mg represents a low dose that may be increased to 3.2 mg based on the safety and PK data. IN dose of 3.2 mg is estimated to be equivalent to IM dose of 0.2 mg, which is still below the range of approved IM dosage for anaphylaxis of 0.3-0.5 mg.

PK study is conducted in a stepwise manner with a starting lower dose, which may be increased if there are no serious adverse events (SAEs) and low exposure while constant and careful monitoring are maintained throughout the study by experienced clinical team including an allergy expert.

FMXIN002 is investigated in a single administration. The safety and tolerability of FMXIN002 can be based on the evidence from published literature and animal studies. Epinephrine for nasal administration (Adrenaline) is already approved for multiple administrations and available at higher doses as OTC product as well as for use in surgery. The safety of IN epinephrine in adults with seasonal allergies, has also been demonstrated in other studies where the administered dose of epinephrine was higher than the suggested dose in the current investigation of FMXIN002. In the study conducted by Chen et al. (9), the most common (≥5% overall) treatment-emergent adverse events (TEAEs) in the epinephrine nasal spray groups were nasal discomfort, tremor, headache, nasal congestion, rhinorrhea, dermatitis contact, and presyncope. In another clinical study of IN epinephrine in saline formulation in healthy adults, transient tremor was observed in one subject and palpitation in two subjects. Increase in heart rate, and diastolic and systolic blood pressures occurred at Tₘₐₓ in most subjects but no correlation was found between these symptoms and the plasma concentrations of epinephrine. No serious adverse effects were observed in the subjects after IN epinephrine administration. Therefore, a good safety profile of FMXIN002 use is expected.

Comparative bioavailability between the test and reference products and also within treatment comparisons of the test product with and without allergen challenge will be determined by a statistical comparison of the AUCₜ, AUC_{inf}, and Cₘₐₓ parameters for epinephrine.

### Example 6: Epinephrine Bitartrate/Sodium Di-Hydrogen Phosphate Composition with Lactose Monohydrate: High Loading of Epinephrine Bitartrate

Epinephrine bitartrate (5.0 g) and sodium di-hydrogen phosphate (1.5 g) were dissolved for 20 min in 30 g of acetone and 60 g water mixture under nitrogen and stirring at 300 rpm. An appropriate size magnetic bar was placed in the receiver and lactose monohydrate (5.0 g) was added thereto, with the stirring rate set at 150 rpm. A clear and homogeneous solution of the drug was obtained, and spray-dried using the modified Büchi Mini Spray-Dryer with inlet nitrogen temperature of 130 °C and outlet temperature of 80 °C, thereby obtaining a dry powder of epinephrine bitartrate/sodium di-hydrogen phosphate, which was further blended in-situ with lactose monohydrate in the receiver. Stirring was maintained in the receiver during the entire process. The loading of epinephrine in the composition was about 8.6% w/w.

The obtained epinephrine bitartrate/sodium di-hydrogen phosphate composition was subjected to particle size analysis using a Malvern Laser Diffraction instrument. As shown in Table 3 the following particle size distribution was obtained: D (10)=8.3 μm, D (50)=81.4 μm and D (90)=151 μm. The amount of the obtained particles having the size less than 10 μm was about 8.2% v/v. 5 μm was about 5.0% v/v.

### Example 7: Epinephrine Bitartrate Drug-Device Combination Product Preparation of Dose 3.6 Mg Epinephrine

Aptar Unit-Dose Powder disposable devices, assembled according to the manufacturer's guidelines, were filled with the epinephrine bitartrate/sodium di-hydrogen phosphate composition prepared in Example 6. Each device contained 42 mg of powder including 3.6 mg of epinephrine.

### Example 8: Epinephrine Bitartrate Drug-Device Combination Product Preparation of Dose 4.0 Mg Epinephrine

Aptar Unit-Dose Powder disposable devices, assembled according to the manufacturer's guidelines, were filled with the epinephrine bitartrate/sodium di-hydrogen phosphate composition prepared in Example 6. Each device contained 47 mg of powder including 4.0 mg of epinephrine.

### Example 9. PH of the Drug-Device Composition

pH was measured as 1% in water and as found in the range of 4-7 for multiple batches.

### Example 10. Particle Size Distribution of Epinephrine Microspheres Emitted from the Device

About 10 devices were shot into a stirred 2000 rpm Isopar G media. PSD was measured using a Malvern Laser Diffraction instrument. As shown in Figure XX, the following particle size distribution was obtained: D (10)=8.74 μm, D (50)=69.2 μm, and D (90)=1 27 μm. The amount of particles smaller than 10 μm was about 11% v/v. The amount of particles having a size of 5 μm was 6% v/v. The particle size distribution of epinephrine microspheres thus meets powder Bulk Drug Product specification and safety requirements.

The comparison between the PSD of the bulk powder loaded into the device and that of the drug constituent released from the device is summarized in Table 3.

TABLE 3
PSD of Bulk powder vs powder released from devices

| Item/ | | | |
|---|---|---|---|
| Material | D(10 μm | D(50) μm | D(90) μm |
| Bulk powder | 8.3 | 81.4 | 151 |
| of FIG. 4 | | | |
| Powder | 8.7 | 69.2 | 127 |
| released | | | |
| from the | | | |
| device | | | |

As shown in Table 3, no significant changes occurred during the filling and assembly of the devices, or during device actuation by shot. The particle size distribution remained similar in both cases. Two populations of particles, including small (10-40 microns) particles of buffered epinephrine and large 50-200 microns particles of lactose monohydrate carrier, are clearly seen in FIG. 5.

### LIST OF REFERENCES

1. Reber, L. L., Hernandez, J. D., Galli, S. J. The pathophysiology of anaphylaxis. J Allergy Clin Immunol. 2017; 140 (2): 335-348
2. Turner, P. J., Jerschow, E., Umasunthar, T., Lin, R., Campbell, D. E. and Boyle, R. J. Fatal Anaphylaxis: Mortality Rate and Risk Factors. J Allergy Clin Immunol Pract. 2017; 5 (5): 1169-1178.
3. Kemp, S. F., Lockey, R. F., Simons, F. E. and World Allergy Organization ad hoc Committee on Epinephrine in, A. Epinephrine: the drug of choice for anaphylaxis—a statement of the world allergy organization. World Allergy Organ J. 2008; 1 (7 Suppl): S18-26.
4. Sicherer, S. H. and Simons, F. E. R. Epinephrine for First-aid Management of Anaphylaxis. Pediatrics. 2017a; 139 (3).
5. Sicherer, S. H., Simons, F. E. R., Section On, A. and Immunology. Epinephrine for First-aid Management of Anaphylaxis. Pediatrics. 2017b; 139 (3).
6. Ring, J., Beyer, K., Biedermann, T., Bircher, A., Duda, D., Fischer, J., et al. Guideline for acute therapy and management of anaphylaxis; Allergo J Int. 2014; 23 (3): 96-112
7. Mylan Inc., Highlights of Prescribing Information. EPIPEN® (epinephrine injection, USP). USA: FDA; Revised: August 2018. https://www.accessdata.fda.gov/scripts/cder/daf/. Accessed [Aug. 6, 2019]
8. Gold M S, Sainsbury R. First aid anaphylaxis management in children who were prescribed an epinephrine autoinjector device (EpiPen). J Allergy Clin Immunol. 2000; 106:171-6
9. Chen, J., Yu, J., Chilampalli, C., DeCastrol, G., Narayanan, E., Wakaskar, R., et al. An Open-Label, 5-Treatment, Crossover, Single-Dose Pharmacokinetic Study of Epinephrine Nasal Spray in Comparison to EpiPen® Intramuscular Injection in Healthy Adults With Seasonal Allergies (abstract 434). AAAAI Annual Meeting. San Francisco, CA, USA, INSYS Development Company, Inc. 2019
10. Cady R K, McAllister P J, Spierings E L, et al. A randomized, double-blind, placebo-controlled study of breath powered nasal delivery of sumatriptan powder (AVP-825) in the treatment of acute migraine (The TARGET Study). Headache. 2015; 55 (1): 88-100. doi: 10.1111/head.12472
11. Orgel H A, Meltzer E O, Bierman C W, Bronsky E, Connell J T, Lieberman P L, Nathan R, Pearlman D S, Pence H L, Slavin R G, et al. J Allergy Clin Immunol. 1991 August; 88 (2): 257-64
12. Food and Drug Administration. FYs 2013-2017 Regulatory Science Report: Locally-Acting Orally Inhaled and Nasal Drug Products. OGD FY13-FY17 Regulatory Science Research Report—1; February 2018. https://www.fda.gov/drugs/generic-drugs/fys-2013-2017-regulatory-science-report-locally-acting-orally-inhaled-and-nasal-drug-products. Accessed [Nov. 20, 2019]
13. Nasal Spray and Inhalation Solution, Suspension, and Spray Drug Products-Chemistry, Manufacturing, and Controls Documentation; Guidance for Industry; CDER, July 2002

1. A dry powder pharmaceutical composition for intranasal administration comprising epinephrine or a pharmaceutically acceptable salt thereof and a pharmaceutically acceptable carrier, wherein the pharmaceutical composition is characterized in that the composition, when emitted from an intranasal powder delivery device, has an emitted particle size distribution with an emitted Dv90 of less than about 400 microns.

2. The dry powder pharmaceutical composition of claim 1, wherein the composition has an emitted particle size distribution with an emitted Dv90 of less than about 200 microns.

3. The dry powder pharmaceutical composition of claim 1, wherein the composition has an emitted particle size distribution with an emitted Dv50 between about 25 microns and about 200 microns.

4. The dry powder pharmaceutical composition of claim 1, wherein the composition has an emitted particle size distribution with an emitted Dv10 between about 5 microns and about 25 microns.

5. The dry powder pharmaceutical composition of claim 1, wherein the composition has an emitted particle size distribution that is essentially the same as a bulk particle size distribution.

6. The dry powder pharmaceutical composition of claim 5, wherein the bulk particle size distribution is characterized by a bulk Dv10 of about 5 μm to about 25 μm, a bulk Dv50 of about 25 μm to about 200 μm, and a bulk Dv90 of less than about 400 μm.

7. The dry powder pharmaceutical composition of claim 1, wherein the composition comprises: a first type of solid particles comprising epinephrine or a pharmaceutically acceptable salt thereof and a physiologically acceptable buffering agent; and a second type of solid particles comprising a pharmaceutically acceptable carrier.

8. The dry powder pharmaceutical composition of claim 7, wherein the first type of solid particles comprises epinephrine bitartrate and sodium dihydrogen phosphate, and wherein the second type of solid particles comprises lactose monohydrate.

9. The dry powder pharmaceutical composition of claim 7, wherein at least about 90% of the first type of solid particles have a mean particle size of about 10 μm to about 40 μm, and wherein the second type of solid particles have a mean particle size of about 50 μm to about 200 μm.

10. The dry powder pharmaceutical composition of claim 1, wherein a single dose of the dry powder pharmaceutical composition comprises between about 1.6 mg and about 5.5 mg of epinephrine or the pharmaceutically acceptable salt thereof.

11. A dry powder pharmaceutical composition for intranasal administration comprising epinephrine or a pharmaceutically acceptable salt thereof and a pharmaceutically acceptable carrier, wherein the pharmaceutical composition is characterized in that the composition, when emitted from an intranasal powder delivery device, has an emitted particle size distribution that is essentially the same as a bulk particle size distribution.

12. The dry powder pharmaceutical composition of claim 11, wherein the bulk particle size distribution and the emitted particle size distribution is characterized by an emitted Dv10 that is essentially the same as bulk Dv10.

13. The dry powder pharmaceutical composition of claim 11, wherein the bulk particle size distribution and the emitted particle size distribution is characterized by an emitted Dv50 that is essentially the same as bulk Dv50.

14. The dry powder pharmaceutical composition of claim 11, wherein the bulk particle size distribution and the emitted particle size distribution is characterized by an emitted Dv90 that is essentially the same as bulk Dv90.

15. The dry powder pharmaceutical composition of claim 11, wherein the bulk particle size distribution and the emitted particle size distribution is characterized by a Dv10 of about 5 μm to about 25 μm, a Dv50 of about 25 μm to about 200 μm, and a Dv90 of less than about 400 μm.

16. The dry powder pharmaceutical composition of claim 11, wherein a single dose of the dry powder pharmaceutical composition comprises between about 1.6 mg and about 5.5 mg of epinephrine or the pharmaceutically acceptable salt thereof.

17. A dry powder pharmaceutical composition for intranasal administration comprising epinephrine or a pharmaceutically acceptable salt thereof and a pharmaceutically acceptable carrier, wherein the pharmaceutical composition is characterized in that the composition, when emitted from an intranasal powder delivery device, has an emitted particle size distribution with an emitted Dv50 between about 25 microns and about 200 microns and an emitted Dv10 between about 5 microns and about 25 microns.

18. A dry powder pharmaceutical composition for intranasal administration comprising epinephrine or a pharmaceutically acceptable salt thereof and a pharmaceutically acceptable carrier, wherein the pharmaceutical composition is characterized in that the composition is substantially free of agglomerated particles and retains at least 80% of initial drug assay after storage for 3 months at 40 °C with 75% relative humidity.

19. A method of treating anaphylaxis or anaphylactic shock in a subject in need thereof, comprising intranasally administering to the subject a therapeutically effective amount of the dry powder pharmaceutical composition of claim 1.

20. A method of intranasally delivering epinephrine to a subject in need thereof, comprising actuating an intranasal powder delivery device comprising the dry powder pharmaceutical composition of claim 1.
